(19) United States
(12) Patent Application Publication
(10) Pub. No.: US 2009/0202509 A1
(43) Pub. Date: Aug. 13, 2009

(54) METHODS AND AGENTS FOR REDUCING OXIDATIVE STRESS

(75) Inventor: Xavier Leverve, La Terrasse (FR)

Correspondence Address: MCANDREWS HELD & MALLOY, LTD, 500 WEST MADISON STREET, SUITE 3400, CHICAGO, IL, 60661, US

(73) Assignee: ETREN, Kremllin Bicetre (FR)

(21) Appl. No.: 12/280,749

(22) PCT Filed: Mar. 1, 2007
(86) PCT No.: PCT/EP07/51966

§ 371 Date: Nov. 11, 2008

(30) Foreign Application Priority Data
Mar. 1, 2006 (GB) 0603975.4

(51) Int. Cl. A61K 38/44; A61P 25/28

(52) U.S. Cl. 424/94.4

(57) ABSTRACT

The present invention relates to novel methods for enhancing endogenous protection mechanisms against oxidative stress, and agents for use in such methods. In particular, the present invention provides a pharmaceutical composition which provides an oxidative signal upon administration to a subject, the signal triggering a therapeutic or prophylactic effect by priming the subject's body to combat the effects of oxidative stress.

The present invention relates to novel methods for enhancing endogenous protection mechanisms against oxidative stress, and agents for use in such methods.

Oxidative stress is a general term used to describe the damage caused to a cell, tissue or organ induced by a reactive oxygen species (ROS). Reactive oxygen species represent a class of molecules that are derived from the metabolism of oxygen and which inherently exist in all aerobic organisms. ROS are either free radicals, reactive anions containing oxygen atoms, or molecules containing oxygen atoms that can either produce free radicals or which are chemically activated by free radicals. Examples of ROS include hydroxyl radicals, superoxide radicals, hydrogen peroxide, and peroxynitrites.

There are many different sources from which reactive oxygen species can be generated, including radiation, UV light, and certain compounds referred to as redox cycling agents, which include some pesticides. In addition, humans are constantly exposed to environmental ROS, in the form of smog, tobacco smoke and other environmental toxins. Most ROS arise endogenously, however, as by-products of normal and essential metabolic reactions, such as energy generation from mitochondria or de-toxification reactions in the liver, for example, utilizing the cytochrome P-450 enzyme system.

Most of the systems for the production of ROS produce superoxide radicals (O₂. ′) or/and hydrogen peroxide (H₂O₂). Studies have also suggested the possibility that superoxide radicals and hydrogen peroxide could interact with each other to produce more reactive hydroxyl radicals (.OH) in the presence of certain metals, particularly free iron or copper ions. Superoxide can also react with nitric oxide to produce peroxynitrate (OONO⁻), another highly reactive oxidizing molecule.

Damage to cells as a result of ROS occurs because of ROS-induced alteration of macromolecules such as polyunsaturated fatty acids in membrane lipids, proteins, and DNA. For example, the amino acids cysteine, methionine, and histidine are especially sensitive to attack and oxidation by the hydroxyl radical. ROS-induced oxidation of proteins can lead to changes in the proteins' three-dimensional structure as well as to fragmentation, aggregation, or cross-linking of the proteins. Protein oxidation will also often make the marked protein more susceptible to degradation by cellular systems responsible for eliminating damaged proteins from the cell. Phospholipids are essential components of the membranes that surround the cells as well as other cellular structures, such as the nucleus and mitochondria. Damage to the phospholipids by ROS thus compromises the viability of the cells. Polyunsaturated fatty acids present in membrane phospholipids are particularly sensitive to attack by hydroxyl radicals and other oxidants. Further to this, ROS are a major source of DNA damage, causing strand breaks, removal of nucleotides, and a variety of modifications of the organic bases of the nucleotides.

Aerobic organisms exhibit physiological and biochemical adaptations to minimize the damaging effects of ROS. Under normal conditions, ROS are cleared from the cell by the actions of antioxidants superoxide dismutase (SOD), catalase, and/or glutathione (GSH) peroxidase.

SODs are metal-containing enzymes that are dependent upon a bound manganese, copper or zinc for their antioxidant activity. SOD catalyzes the reduction of superoxide anions to hydrogen peroxide, which is substantially less toxic than superoxide, and oxygen. Catalase is primarily found in peroxisomes, and degrades hydrogen peroxide to water and oxygen, thereby completing the detoxification reaction. Glutathione peroxidase constitutes a group of enzymes, the most abundant of which contain selenium. These enzymes, like catalase, degrade hydrogen peroxide. They also reduce organic peroxides to alcohols, providing another route for eliminating toxic oxidants.

Oxidative stress occurs when the level of ROS exceeds a system's ability to clear them. This imbalance can result from a lack of antioxidant capacity caused by a disturbance in production or distribution of antioxidant entities, or by an overabundance of ROS.

Excessive levels of ROS and the resulting oxidative stress have been implicated in a variety of human diseases, including pulmonary conditions; ischemia/reperfusion neuronal injuries; inflammatory diseases such as rheumatoid arthritis or fibrosis; atherosclerosis; degenerative disease of the human temporomandibular-joint; viral processes, such as HIV infection; cataract formation; macular degeneration; degenerative retinal damage; Down's syndrome; liver disease associated with chronic alcohol consumption; non-vascular gastrointestinal disorders; multiple sclerosis; muscular dystrophy and human cancers, as well as damage caused by exposure to UV rays, and the aging process itself.

The detrimental effects of oxidative stress in cardiac tissue are also well documented, and in particular, those that result from ischemia/reperfusion injuries.

Further to this, oxidative stress and ROS have been implicated in neurodegenerative diseases, including Alzheimer's disease (AD), Parkinson's disease (PD) and Lewy body disease. A role of oxidative stress in PD is supported by findings of increased oxidative damage to lipids, DNA and proteins in human post-mortem Parkinsonian brains, and in animal models of PD. Additionally, the catecholamine neurotransmitter dopamine, which is utilized by the cells which degenerate in Parkinson's disease, and a precursor of which is frequently given as a treatment in PD, can undergo oxidation to produce active oxygen species. Markers of oxidative stress have similarly been found both in Alzheimer's disease brain tissue post-mortem, and in peripheral blood from patients with AD.

Impaired levels of SOD have also been found in brain areas involved in Parkinson's, and Alzheimer's disease. Studies have also demonstrated lower levels of SOD in certain brain areas of people affected by Alzheimer's disease. Oxidative stress has also been implicated in amyotrophic lateral sclerosis (ALS), a fatal neurogenerative disorder characterized by degeneration of upper and lower motor neurons.

Following an increase in the understanding of the damaging effects of oxidative stress, the mechanisms underlying its causes and the role of antioxidants in limiting the effects of ROS, methods of increasing endogenous levels of free radical scavengers, or antioxidants, have been the subject of much consideration.

Many food groups are known to be high in antioxidants, for example, tomatoes, citrus fruit, green vegetables, carrots and black tea. Further to this, the benefits of ingested antioxidants in isolation, such as tablets of vitamin C and E, beta-carotene, ubiquinone, bioflavonoids and phenolic acid, as well as glutathione and SOD themselves have been examined. There are several reports of the benefits of vitamin E, in particular, in various conditions in which oxidative stress is implicated, including Parkinson's disease and Alzheimer's disease and ALS. However, there are also numerous reports on the limited or lack of improvement seen in such patients when treated with oral antioxidants, and this is frequently attributed to the fact that the ingestion of antioxidants is highly unlikely to result in an increase in circulating levels, due to breakdown of the antioxidant during the digestive processes.

Recently, attempts have been made to overcome the problems associated with orally administered SOD. The intention is to increase the intracellular levels of SOD in the body, and to thereby increase the conversion of superoxide to hydrogen peroxide. However, SOD, like other proteins, will be broken down by the digestive process when administered orally.

U.S. Pat. No. 6,045,809 discloses administration of a combination of SOD and a lipid, preferably selected from the group consisting of ceramides, phospholipids, tylacoids and diacylglycerols or a protein, preferably selected from the group comprising prolamines and polymer films based on prolamines (for example, gliadin) with, optionally, a pharmaceutically acceptable vehicle. U.S. Pat. No. 6,045,809 alleges that such combinations have improved passage of the SOD through the digestive system and result in higher plasma concentrations. However, the effect of the lipids is unclear. It is possible that the lipids are, to a limited extent, having a protective effect, shielding the SOD from degradation. However, it would appear that the increased absorption of SOD is actually due to an effect of certain specific lipids which are able to create a path through the mucus of the intestine wall, thereby increasing the likelihood of the administered SOD reaching the intestine wall. In Clemente et al. (Gut. 2003 February; 52(2):218-23), there is provided an explanation of how a gliadin coating could increase circulating levels of an element, such as SOD, contained within it. This paper explains that gliadin increases intestinal permeability, presumably allowing the coated element to be rapidly transported through the intestinal wall and into the bloodstream.

Novus Research Products have developed the ‘nutraceutical product’ Glisodin®. Glisodin® is a water-soluble form of plant SOD extract from Cucmis melo LC (melon), chemically combined with a wheat gliadin biopolymer system. In promotional material, Glisodin® is said to increase circulating levels of SOD. However, whilst tests in humans using the Comet assay to assess DNA strand breakages revealed that orally administered Glisodin® protected against DNA damage following an episode of oxidative stress, no significant changes in blood SOD activity were found following oral consumption of 1000 UI-NBT per day (Muth et al., Free Radical Res. 2004. 38(9): 927-932).

A study by Nelson et al. (Free Radical Bio. Med. 2006. 40: 341-347) aimed to decrease oxidative stress by inducing SOD and catalase production through administration of plant extracts. Levels of lipid peroxidation products were assessed in healthy volunteers before and after receiving daily supplements of Protandim® (Lifeline Therapeutics Inc, Denver, Colo., USA) using TBARS (thiobarbituric acid-reactive substances). Protandim® comprises five botanical ingredients (B. monniera, S. marianum, W. somnifera, C. sinensis and C. longa) said to increase the activities of SOD or catalase whilst decreasing TBARS. Results showed a decrease in TBARS following prolonged dosing, and an increase in erythrocyte SOD and catalase. However, there is no demonstration in Nelson et al. of any increase in SOD or catalase levels in tissues.

Furthermore, the plasma half-life of SOD, although variable between different types, is known to be as little as 4-6 minutes. Accordingly, any increase in the circulating concentration of SOD resulting from ingestion of products such as Glisodin®, or those disclosed in Nelson will be transient.

It has been shown that adaptive protection against subsequent oxidative damage can be triggered by prior activation of the endogenous mechanisms for dealing with oxidative stress. For example, studies in spontaneously hypertensive rat (SHR) hearts, which provide a model for hypertension in humans, have shown that although the SHR hearts were more sensitive to ischemia/reperfusion and generated more ROS during reperfusion than normotensive control hearts, pre-conditioning induced by the ROS to be released during oxidative stress improved the post-ischaemic recovery of myocardium function (Csonka et al., Free Rad. Biol & Med. 2000. 29(7): 512-619).

Additionally, studies have shown that a single hyperbaric oxygen treatment in human subjects, serving as an in vivo model for the instigation of oxidative stress, triggers oxidative adaptive protection against DNA damage (Rothfuss et al., Carcinogenesis. 1998. 19(11): 1913-7). Interestingly, in this context, despite the impaired pulmonary diffusion capacity due to cumulative hyperoxia resulting from the repetitive exposures to increase inspired oxygen partial pressures that active scuba divers (>37 dives/year) present with, which includes increased formation of oxygen radicals, pre-existing diving-associated episodes of hyperoxia are thought to induce a degree of protection against subsequent hyperbaric induced oxidative stress.

Further to this, studies in vitro have also shown that treatment of a variety of healthy cell cultures with hydrogen peroxide as a direct mediator of oxidative stress, or with redox cycling compounds paraquat or menadione, leads to an increase in catalase and MnSOD mRNA levels (Bai et al., J. Bio. Chem. 1999. 274: 26217-26224; Röhrdanz et al., Brain Research. 2001. 900: 128-136)

Clearly, it would be advantageous, in both the treatment of conditions associated with oxidative stress, and in order to improve the scavenging of ROS in non-pathological situations as an aid to general well being, to ‘up-regulate’ the body's own defence mechanisms against ROS, and the studies discussed above provide a basis for doing this.

However, the disadvantage of the above discussed studies and other known methods for triggering a protective ‘up-regulation’ of the mechanisms utilized endogenously to eliminate ROS, is that they require exposure to an exogenous factor which results in oxidative stress. This is clearly not acceptable, particularly in pathological situations where cellular function is already compromised, for example, in human disease conditions associated with oxidative stress, where exposure to such a stress could further disease progression and/or cause a transient, prolonged or permanent exacerbation of symptoms.

Alternative methods of enhancing endogenous ROS defence methods are, therefore, required.

It is now hypothesized that, because one of the causes of the imbalance between antioxidants and ROS that can lead to oxidative stress, as discussed above, is a disturbance in the production or distribution of antioxidant entities, disruption of levels and functioning of the antioxidant entity SOD in diseases such as Parkinson's disease, Alzheimer's disease and ALS impacts upon the cellular concentration of H₂O₂. A decrease in H₂O₂ levels will affect downstream pathways, including signalling, scavenging and peroxidizing pathways, with detrimental repercussions.

An object of the present invention is, therefore, to produce an increase in the intracellular concentration of H₂O₂ as a means of up-regulating endogenous oxidative scavenging mechanisms (catalase & glutathione peroxidase (GPX)), and protecting downstream pathways in which H₂O₂ is involved.

According to a first aspect of the present invention, a composition is provided, the composition providing an oxidative signal upon administration to a subject, which triggers a therapeutic or prophylactic effect by priming the subject's body to combat the effects of oxidative stress.

Thus, the invention is based upon the use of a signal of oxidative stress, rather than oxidative stress itself.

Accordingly, administration of compositions according to the present invention does not increase or substantially increase levels of ROS and/or does not increase or substantially increase levels of oxidative stress.

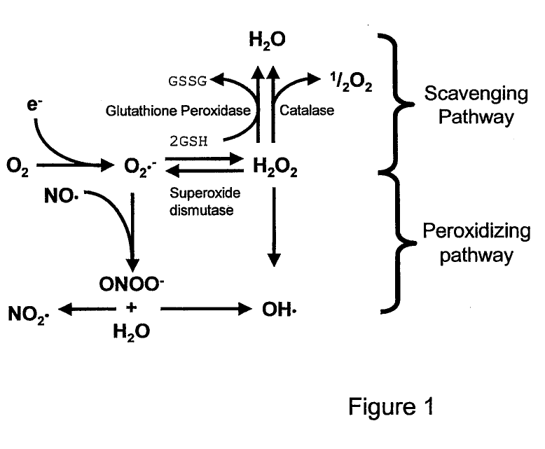

As discussed above, and as can be seen from FIG. 1, the elimination of free radicals by the body is a multi-faceted process, involving several different enzymes and intermediary substrates, of which SOD is just one component. Another component is hydrogen peroxide (H₂O₂).

H₂O₂ is an oxidating compound, which decomposes to water and oxygen, a reaction which is catalysed, as discussed above, by gluthathione peroxidase and catalase. At high concentrations H₂O₂ is cytotoxic. However, unlike superoxide, H₂O₂ does not oxidize most biological molecules readily, including lipids, DNA and proteins (unless the latter have hyper-teactive thiol groups or methionine residues). The danger of H₂O₂ largely comes from its ready conversion to hydroxyl radicals when in the presence of metal ions, such as copper or iron.

Research in recent years has demonstrated that H₂O₂, rather than being just an intermediary substrate in the process of elimination of ROS, also actually plays an important role within the cell. H₂O₂ can cross the cell and mitochondrial membrane, and is now known to act as an intracellular second messenger, which can activate, for example, kinase cascades and transcription factors such as NF_{κ}B and AP-1, affecting processes such as regulation of vascular tone, sensing of oxygen tension, and enhancement of membrane receptor signal transduction. H₂O₂ therefore plays an important role in cell signalling.

Due to the known cytotoxicity of H₂O₂ itself, and also the production of hydroxyl radicals as a result of reduction of H₂O₂ in the presence of metal ions, increasing the intracellular H₂O₂ levels is contraindicated both in healthy subjects, and particularly in patients suffering from a disease in which oxidative stress is involved. However, the increase produced by the present invention will result in an intracellular H₂O₂ concentration that is greater than the minimum concentration arising as a result of a pathological deficit in H 0, and less than a concentration that causes cell toxicity. As such, the present invention is safe for use in all subjects. Unlike other treatments aimed at enhancing endogenous protection mechanisms for dealing with ROS, increasing intracellular H₂O₂ as claimed by the invention hereby does not inflict or cause an oxidative stress itself.

Thus, according to a particularly preferred embodiment of the present invention, the oxidative signal produced by the composition is hydrogen peroxide. As such, the present invention will confer adaptive protection against oxidative stress, wherein endogenous mechanisms for dealing with the factors associated with oxidative stress are up-regulated, without inflicting an oxidative stress itself. The present invention will also enhance H₂O₂ levels in subjects with sub-normal levels, thus restoring the advantages conferred by H₂O₂ as a signalling agent.

The half-life of H₂O₂ varies dramatically according to its environment. As would be understood by the person skilled in the art, the half-life of hydrogen peroxide is strongly dependent upon the presence of transition metal ions such as iron or copper. When hydrogen peroxide is not in contact with metal ions it has a very considerable half-life. However, the smallest trace of metal prompts rapid degradation of H₂O₂ via the Fenton reaction, which can shorten the half-life to less than a second.

Due to methodological limitations, measurement of the small, albeit metabolically substantial, increases in intracellular H₂O₂ concentration produced by the present invention is not possible. However, the effects derived from such increases may be defined in functional and qualitative terms.

For example, the mean life expectancy of an ALS patient from the time of diagnosis is 44 months. Progression of disease state in patients suffering from ALS can be characterized by a loss of motor neurone activity resulting in a decrease in almost any motor activity: skeletal muscle function (legs, arms, mobility), respiratory muscle (breathing, cough), cranial nerve function (speech, swallowing, ocular motricity). Notable stages in the progression of the disease are an impairment in normal oral nutrition, requiring a gastrostomy, and an inability to cough and to breath that may result in death, or a decision to perform a tracheotomy and artificially ventilate the patient. The insertion of a gastric feeding tube most often announces a fatal event within 12-18 months. However, an ALS patient treated according to the present invention is still alive 4 years following insertion of a gastrostomy tube. Since then, the patient has shown no worsening of clinical symptoms, and hence no progression of the disease. Furthermore, clear improvement in clinical state has been achieved, as the patient is no longer reliant upon gastrostomy feeding.

It is well known that immuno-competent cells can induce an oxidative stress (oxidative burst), which results from plasma membrane NADPH oxidase, which is responsible for superoxide generation from NADPH oxidation and oxygen (see, for example, Schulze-Osthoff et al., “Oxidative stress, antioxidant defenses, and damage removal, repair, and replacement systems” IUBMB Life 2000. 50(4-5):279-89; Davies “Oxidative stress and signal transduction” Int. J. Vitam. Nutr. Res. 1997. 67(5):336-42; Guzik et al., “Nitric oxide and superoxide in inflammation and immune regulation” J. Physiol. Pharmacol. 2003. 54(4):469-87). The generated superoxide ions are then converted to H₂O₂ in the reaction catalysed by SOD. Induction of oxidative stress and the resultant generation of hydrogen peroxide appears to be the common response of immune cells upon activation.

Approximately 80% of immuno-competent cells are contained in the gut and they are present in the gut wall in specific areas called “gut associated lymphoid tissue” (GALT). These immuno-competent cells, after a stay in the intestinal wall, are released from the gut via the lymphatic system, and circulate within the whole body. Information obtained during their stay in the gut can be diffused to every tissue.

Immuno-competent cells are also found outside of the gut, for example in the skin, genital tract and lung, as well as in the blood.

Oxidative signalling produced by the compositions of the invention following oral administration is transmitted to immuno-competent cells in the gut wall. These cells subsequently transmit the signal, either through transmission, or delocalisation. This has been shown both in animal studies and in human beings.

The signalling of an oxidative stress to the immuno-competent cells may also be produced by other means, for example by exposure of the subject to hyperbaric oxygen or by intravenous injection of an entity capable of creating the same oxidative stress in the form of H₂O₂.

Thus, in one embodiment of the present invention, the oxidative signal is provided to immuno-competent cells in the gut, more specifically to immuno-competent GALT cells. Alternatively, the oxidative signal is provided to immuno-competent cells elsewhere in the subject's body. The signal is preferably in the form of hydrogen peroxide.

Oral ingestion, topical application or injection of diluted solutions of hydrogen peroxide is advocated by some health groups. The purpose of such treatments is to increase levels of oxygen within the body, as the H₂O₂ is rapidly decomposed. However, the Agency for Toxic Substances and Disease Registty state that hydrogen peroxide is not absorbed by the skin, and that ingestion of even a weak solution can cause gastric irritation, vomiting and diarrhoea, with higher concentrations causing systemic toxicity, which has been associated with fatalities. Side effects associated with the injection of weak solutions of hydrogen peroxide include faintness, fatigue, headaches and chest pain with risk of pulmonary oedema and death at higher concentrations. Further to this, ingested H₂O₂ rapidly decomposes in the digestive system, and is therefore not absorbed into the blood stream. The average half-life of H₂O₂ in human blood is reported to be 0.75 seconds. As a result, oral ingestion, topical application or injection of hydrogen peroxide will not increase intracellular levels of H₂O₂.

Accordingly, in a preferred embodiment of the present invention, a means is provided of increasing the intracellular concentration of hydrogen peroxide in a mammalian subject, for the purpose of providing protection against oxidative stress.

In certain embodiments, the intracellular H₂O₂ concentration is raised by the administration of an agent.

The agent may be administered by any conventional route, including orally, nasally, or by inhalation or injection.

Agents which may advantageously be used in the present invention include nicotinamide adenosine di-nucleotide phosphate (NADPH), NADH, substrates of SODs, such as superoxide anions; mitochondrial substrates, such as succinate, choline, proline, malate, pyruvate, ketoglutarate or glycerol 3-phosphate; phorbol myristate acetate; factors involved in H₂O₂ production such as antimycin A, antimycin, various quinones such as ubiquinone, rotenone; glycollate oxidase, D-amino acid oxidase, monoamine oxidases; SODs; oxidised natural anti-oxidants such as oxidised flavonoids, or oxidised vitamins such as oxidised vitamin C or oxidised vitamin E; or combinations of the foregoing.

In preferred embodiments, the agent is a SOD.

In embodiments wherein the agent is a SOD, it is preferred that the SOD is derived from yeast or wheat.

In some embodiments of the present invention, compositions may additionally comprise one or more of, naturally occurring oligosaccharides, preferably of vegetable origin, such as those found in food such as seed husks or shells, prolamines, preferably of vegetable origin and derived from at least one cereal selected from the group consisting of wheat, rye, barley, oats, rice, millet and maize, for example gliadin from wheat, or polymer films derived from such prolamines.

In some embodiments, compositions may additionally comprise one or more gastroresistant ingredients, such as those well known in the art of orally administered therapeutic agents.

In a preferred embodiment of the present invention, the composition comprises one or more agents in combination with gliadin, or a naturally occurring oligosaccharide and a gastroresistant ingredient.

In some embodiments, compositions according to the present invention may additionally comprise one or more pharmaceutically acceptable excipients or carriers, which may be incorporated in order to improve the stability of the composition, one or more ions such as zinc, copper, magnesium, selenium or manganese in nutritional proportions, and/or one or more neurotransmitters, such as dopamine.

In one embodiment of the invention, the agent is not SOD, is not a combination of SOD and gliadin, or does not comprise SOD.

Orally administered SOD is thought to produce an oxidative signal by generating hydrogen peroxide from superoxide. Thus, it is proposed that oral administration of SOD leads to an increase in the concentration of intraluminal SOD. This intraluminal SOD is active through its product, H₂O₂, which is known to cross the cell and mitochondtial membrane.

In addition, the oxygen content in the intraluminal fluid decreases from upstream to downstream. In the distal ileum and in the colon, the oxygen content is very low (anaerobic microflora). Meanwhile, due to the active bacterial metabolism, there are numerous redox reactions. Therefore, a small and limited amount of superoxide is being produced (as superoxide only originates from oxygen). This explains why even a high concentration of intraluminal SOD does not lead to a toxic effect. The rate of H₂O₂ production is being limited by oxygen content.

It is important for the activity of SOD to be limited by the limited superoxide substrate, otherwise the increase in SOD concentration can have a deleterious effect, dangerously increasing the production of H₂O₂ in an environment that might not contain a relevant corresponding concentration of catalase or glutathione peroxidase, therefore increasing the likeliness of the Fenton effect, which is more dangerous than the presence of the superoxide.

The inventors have obtained evidence of an anti-oxidant effect in the brain of rats following the oral administration of a composition comprising SOD and gliadin in a dose of 1000 IU/kg of animal weight/day for 3 weeks. The increase in the brain anti-oxidant defence was evidenced by a decreased staining of myeloperoxidase products contrasting with normal protein staining (anti-myeloperoxidase antibodies), which can be explained as an enhanced anti-oxidant defence in the brain (unpublished).

The inventors have also shown that oral administration of SOD-gliadin is responsible for enhanced anti-oxidant capacities in white blood cells in rats and in humans. In addition, the results obtained in the brain where animals were orally administered SOD gliadin support the explanation that enhanced anti-oxidant capacities are being transmitted to peripheral cells, resulting in an enhanced endogenous defence against oxidative stress (unpublished).

Similar results were obtained in humans. Healthy humans received SOD and gliadin (1000 IU/day) or placebo for 2 weeks. After this period, subjects were exposed to hyperbaric oxygen (2.5 atin for 2 hours) and the DNA of immune cells was studied using the comet assay. A significant difference in the DNA image was observed between treated group and those receiving the placebo, the treated subjects exhibiting a less damaged DNA than those who received the placebo (Muth et al., Free Radical Res. 2004. 38(9): 927-932)

In some embodiments, the composition may comprise one or more gastroresistant ingredients, such as those well known in the art of orally administered therapeutic agents, for example, polymers such as cellulose acetate phthalate, cellulose acetate trimellitate, hydroxypropylmethylcellulose phthalate, or Eudragit L and S, lipids, including plant lipids or proteins, including plant proteins. In some embodiments the ingredients may be micronised to achieve gastroresistance characteristics. In alternative embodiments, the gastroresistant ingredients may envelope or substantially coat the agent, so that the ingredients of the composition are effectively encapsulated by the gastro-resistant ingredients. In some embodiments this may allow the agent to be administered without the need for an additional ingredient, such as gliadin, to improve intestinal permeability.

In some embodiments of the present invention, the agent may be combined with one or more pharmaceutically acceptable excipients or carriers, which may be conventional components in pharmaceutical compositions and may be selected depending upon the intended route of administration.

In some embodiments the agent may be combined with one or more ions such as zinc, copper, magnesium, selenium or manganese, in nutritional proportions.

In certain embodiments the agent may be combined with one or more neurotransmitters, such as dopamine. Oxidative deamination of catecholamines such as dopamine, as discussed above, results in H₂O₂ formation. Accordingly, administration of a catecholaminergic neurotransmitter may synergize the effects of a co-administered agent.

In one embodiment the agent may be combined with a prolamine or a naturally occurring oligosaccharide, one or more gastroresistant ingredients, one or more ions, and/or a neurotransmitter, and a pharmaceutically acceptable excipient or carrier.

Preferably, the present invention is used to treat conditions that are associated with oxidative stress. These conditions include those in which oxidative stress is an underlying cause or linked with the underlying cause, as well as conditions where oxidative stress is a symptom or where oxidative stress is caused by the conventional treatment of the condition.

The present invention may, for example, be used for treating conditions including ALS; Parkinson's disease; Alzheimer's disease; cardiac conditions such as cardiac ischemia/reperfusion injuries; pulmonary conditions including pulmonary hypoxic diseases such as those involving chronic hypoxia, such as chronic obstructive pulmonary disease (COPD); neuronal ischemia/reperfusion injuries; inflammatory diseases such as rheumatoid arthritis or fibrosis; atherosclerosis; degenerative disease of the human temporomandibular-joint; viral processes, such as HIV infection; cataract formation; macular degeneration; degenerative retinal damage; Down's syndrome; liver disease associated with chronic alcohol consumption; non-vascular gastrointestinal disorders; multiple sclerosis; muscular dystrophy and human cancers.

In alternative embodiments, the present invention may be administered prophylactically to subjects with a pre-disposition to a condition associated with oxidative stress.

In an especially preferred embodiment of the invention, the compositions are for treating ALS (including FALS), Alzheimer's disease, Parkinson's disease, Down's syndrome, pulmonary conditions including pulmonary hypoxic diseases such as those involving chronic hypoxia, for example chronic obstructive pulmonary disease (COPD), and neuronal ischemia/reperfusion injuries.

Amyotrophic lateral sclerosis (ALS) is a fatal neurogenerative disorder characterized by degeneration of upper and lower motor neurons. A growing body of evidence indicates that mitochondrial dysfunction in particular, may play a role in the pathogenesis of ALS, although the mechanisms underlying such dysfunction are largely unknown. Morphological abnormalities of mitochondria (swelling) occurs very early in mice with ALS, and several reports have demonstrated a decrease in mitochondrial DNA as well as in respiratory chain enzyme activities both in ALS patients and in ALS transgenic mice (see Dupuis et al., FASEB. J. Online publication, Sep. 18, 2003).

Whilst most cases of ALS occur sporadically, a familial form of ALS (FALS), which accounts for approximately 20% of all familial cases is known to be caused by a mutation in Cu,Zn-superoxide dismutasel (Cu,Zn-SOD1), which has an effect on the body's anti-oxidant defence. It is thought that the mutations in Cu,Zn-SOD1 cause impaired protein folding, resulting in diminished or altered activity of the Cu,Zn-SOD1 molecule. However, the exact mechanisms underlying this familial form of ALS are not fully understood, and several theories abound.

Studies by Dupuis et al., (FASEB. J. Online publication, Sep. 18, 2003) investigated the expression of mitochondrial uncoupling proteins (UCPs) in tissues from a mouse model of ALS. UCPs are members of the family of mitochondrial carrier proteins. It is thought that UCPs might have a function in the fine regulation of mitochondrial respiration and via this function provide resistance to oxidative stress. In the investigations of Dupuis et al., it was found UCP3 in particular was up-regulated in ALS skeletal muscle from both an animal model (FALS-linked Cu,Zn SOD1 mutation G86R in mice) and human biopsies. UCP3 has been shown to be involved in oxidative stress-inducible proton conductance. UCP2 and 3 are also known to trigger mitochondrial uncoupling both in vivo and in vitro, and this is activated, under physiological conditions of oxidative stress, by superoxide anions, thus limiting ROS production by the mitochondrial respiratory chain. This in turn decreases superoxide levels and UCP uncoupling activity by a feedback loop. Dupuis et al. concluded that it was likely that the up-regulation of UCP3 seen in muscle occurred as a response to high levels of ROS in ALS. Whilst the data did not conclusively demonstrate mitochondrial uncoupling, the chosen interpretation is favoured as a result of the associated decrease in the respiratory control ratio, decreased levels of ATP, and the hallmark mitochondrial swelling that occurs in ALS.

Further studies by Dupuis et al., (PNAS. 2004. 101. 11159-11164) arising from the finding of a decrease in the respiratory control ratio in ALS studies, investigated the role of an energy imbalance in ALS, resulting from metabolic perturbations. Investigations in G86R and G93A mouse transgenic ALS lines demonstrated a reduction in adiposity, decreased plasma levels of insulin and increased levels of corticosteroids, providing compelling evidence of defective energy homeostasis.

However, work on the familial ALS Cu,Zn-SOD1 mutant cloned from mice and overexpressed in an in vitro cell line has also shown that the free-radical generating capacity of the mutant SOD when utilizing H₂O₂ as a substrate is enhanced in comparison to wild-type Cu,Zn-SOD1. This is thought to be attributable to a small decrease in the Kₘ value (the concentration of substrate that leads to half-maximal velocity) of the mutant for H₂O₂ (Yim et al., PNAS, 1996. 93. 5706-5714; Yim et al., J. Bio. Chem. 1997. 272. 8861-8863).

The lowered Kₘ of the mutant Cu,Zn-SOD1 enzyme for hydrogen peroxide encourages reversal of the conversion of superoxide to hydrogen peroxide. This has a number of effects.

Firstly, it will lead to an increase in the generation of superoxide, which can promote inactivation of the mutant Cu,Zn-SOD1 enzyme, leading to the release of its metal ions, with further deleterious repercussions, including involvement of Fenton-type site-specific reactions, enhancement of peroxynitrite-mediated tyrosine nitration, and blocking of phosphorylation, leading to impairment of the downstream signal transduction pathway. The consequential elevated production of free radicals may result in a further, cascade production of free radicals originating from anionic radical scavengers such as neurotransmitters like glutamate and taurine, which are thought to exert more specific deleterious effects in motor neurons.

Secondly, the conversion of hydrogen peroxide to superoxide will lead to a reduction in the levels of hydrogen peroxide. The reduced concentration of hydrogen peroxide may not be sufficient to activate the cellular signalling pathway responsible for the transcription of the enzymes involved in the hydrogen peroxide scavenging pathways, primarily catalase and glutathione peroxidase.

Hence, the deleterious consequence of the mutation of the Cu,Zn-SOD1 enzyme is not limited to the enhanced generation of superoxide, an ROS, but also involves the prevention of the up-regulation of the downstream anti-oxidant pathway which ordinarily leads to the safe elimination of hydrogen peroxide. These consequences cannot both be overcome by administering anti-oxidants such as vitamins, as currently proposed. This would explain the reported limited or absent beneficial effects observed following the treatment of ALS suffers with anti-oxidants.

Further to this, whilst hydrogen peroxide can be detoxified into water, as catalysed by catalase and/or gluthathione peroxidase and excreted harmlessly from the cell, H₂O₂ can be reduced to form toxic hydroxyl radicals in the presence of copper or iron (the Fenton reaction). Maintaining a balance between these two hydrogen peroxide excretory pathways depends on several factors, including the concentration and activity of detoxification enzymes, the presence of metal ions, and the concentration of hydrogen peroxide which appears to act as a signalling metabolite. It has therefore been postulated that both the cause, and some of the symptoms of this type of familial ALS may result from the enhancement of the free-radical generating function rather than, or as well as, a reduction of Cu,Zn-SOD1 activity resulting from the mutation.

A 40 year old women, suffering from ALS has been treated by oral administration of SOD-gliadin (500 IU/day). Treatment was introduced when this patient was at a stage of severe dysphagia (naso-gastric tube and enteral feeding), and almost tetraplegic. The treatment of this patient has continued for some 5 years and during this time no further neurological worsening has been noted. In fact, an improvement of dysphagia was observed in such a way that enteral feeding was interrupted after 2 years. The condition has then stabilized. It should be noted that the average life expectancy of a patient at this stage of the disease would ordinarily be less than 2 years.

From different recent studies, both in animal model and human patients, it appears that the mitochondrial disease affects not only the motor neurones but also several other tissues including muscle and liver. The beneficial effect of the present invention would not be limited to the motor neurones, but would reach all tissues as a result of the circulating activated immune cells.

As discussed above, FALS appears to be caused by a specific enzyme mutation. The patho-physiology of other forms of ALS is less clear. However, the strong similarity of the symptoms strongly suggests that other forms of ALS involve oxidative stress abnormalities, perhaps, for instance, undetected SOD polyfotmisms.

Both Alzheimer's and Parkinson's disease are progressive neurodegenerative conditions in which oxidative stress has been implicated. A study by Choi et al. (J Biol. Chem. 2005. 280(12): 11648-55) reported that Cu,Zn-SOD1 is a major target of oxidative damage in AD and PD brains, thereby implicating oxidative damage to SOD1 in the pathogenesis of sporadic AD and PD.

Down's syndrome results from over-expression of chromosome-21 encoded genes, one of which is the Cu,Zn-SOD gene. Some studies have shown that the activity of SOD is elevated in Down's syndrome. However, such studies believe that SOD activity increases disproportionately to enzymes such as glutathione peroxidase, which, as discussed above, are responsible for the degredation of H₂O₂. As a result, oxidative stress occurs, resulting in cellular damage. Substantial epidemiological and in vitro evidence of such chronic oxidative stress is consistently found in individuals with Down's syndrome.

Free radical-mediated oxidative damage has been implicated in neuronal injury resulting from ischemia/reperfusion events. Such events have been shown to result in an increase in protein oxidation, and a decrease in the activity of glutamine synthetase, which is believed to be a critical factor in the resultant neurotoxicity caused by ischemia/reperfusion injuries. Studies have shown that CuZn-SOD confers neuronal protection from damage resulting from ischemia/reperfusion injuries, by inhibiting apoptotic cell death (Kondo et al. J. Neurosci. 1997. 17(11). 4180-9).

As a result of the similar, although different patho-physiological processes, neurodegenerative diseases other than ALS, and conditions resulting in neuronal injury could clearly benefit from the novel approach to oxidative stress treatment provided by the present invention. Intracellular levels of H₂O₂ appear to be a general sensor of oxidative stress responsible for the regulation of the transcription of several enzymes involved in anti-oxidant defence. Hence, producing an oxidative signal by an exogenous tool in order to prime the subject's body to combat the effects of oxidative stress could be beneficial in the treatment or prevention of several diseases involving a defective endogenous anti-oxidant defence.

Hypoxia may be responsible for enhanced ROS production at the level of the mitochondria. Moreover, the cellular oxygen-level sensing system is sensitive to the generation of H₂O₂ from NADPH oxidase. Oxidative stress has been increasingly recognized as playing a central role in the patho-physiology of diseases involving chronic hypoxia, such as chronic obstructive pulmonary disease (Cell Biochem. Biophys. 2005. 43(1):167-88; Treat. Respir. Med. 2005. 4(3):175-200). However, treatment with anti-oxidants such as vitamins has not lead to very convincing results to date. Restoring H₂O₂ levels in immune cells as proposed in the present invention could appropriately modulate the pro/antioxidant response in such conditions.

In certain embodiments, agents for use in compositions according to the present invention may have an activity of 50, 100, 200, 500, 800, 1000, 1200, 1500, 2000, 2200, 2500, 3000, 3500, 4000, 4500, 5000, 5500 or 6000 IU/mg. In preferred embodiments, the agent has an activity of between 100-5000 IU/mg, more preferably, 250-4000 IU/mg, even more preferably, 500-3500 IU/mg, and most preferably 1000-3200 IU/mg.

As discussed above, compositions according to the present invention may comprise one or more agents, together with additional ingredients, such as one or more of naturally occurring oligosaccharides or prolamines, one or more gastroresistant ingredients and/or one or more excipients. As such, the activity of compositions according to the present invention will depend upon the type and amount of ingredients included in the compositions in addition to the agent(s).

In some embodiments, compositions according to the present invention may comprise a minimal dose of 0.5, 1, 10, 50, 100, 200, 500, 1000, 1500, 2000, 2500, 2800, 2900, 3000, 3500, 4000, 4500, 5000, 5500, 6000, 6500, 7000, 8000, 9000, 10000, 10500 or 11000 IU of agent.

In some uses of compositions according to the present invention, the patient may receive a minimal daily dose of 0.5, 1, 10, 50, 100, 200, 500, 1000, 1500, 2000, 2500, 2800, 2900, 3000, 3500, 4000, 4500, 5000, 5500, 6000, 6500, 7000, 8000, 9000, 10000, 10500 or 11000 IU of agent. Preferably the patient receives a minimal daily dose of between 200-4000 IU of agent, more preferably the patient receives a minimal daily dose of 400-3000 IU of agent, even more preferably the patient receives a minimal daily dose of 500-2000 IU of agent and most preferably, the patient receives a minimal daily dose of 900-1200 IU of agent.

It is thought that there are no detrimental effects associated with administration to a patient of a composition according to the present invention comprising higher levels of agent activity than those discussed above. However, it is believed that treatment with compositions with higher levels of agent activity is unlikely to provide any additional benefits to those seen upon administration of the doses discussed.

In some embodiments of the present invention, the activity level of the daily dose administered is constant throughout treatment. In alternative embodiments, the activity of the dose which is administered daily may be altered during treatment. In embodiments of the present invention, compositions comprise an agent in combination with gliadin, and the ratio of agent to gliadin is preferably between 1 and 95 IU of agent per mg of gliadin, more preferably between 5 and 80 IU of agent pet mg of gliadin, even more preferably between 10 and 70 IU of agent per mg of gliadin, even more preferably between 20 and 60 IU of agent per mg of gliadin and most preferably between 30 and 55 IU of agent per mg of gliadin.

Compositions according to the present invention may be taken alone, or in addition to, or conjunction with known or possible treatments for other conditions, including known or possible treatments for conditions associated with oxidative stress. For example, compositions according to the present invention may be taken in addition to known or possible treatments for ALS (including FALS) Parkinson's disease, Alzheimer's disease, Down's syndrome, pulmonary conditions, neuronal ischemia/reperfusion injuries, inflammatory diseases, atherosclerosis, degenerative disease of the human temporomandibular-joint, viral processes, cataract formation, macular degeneration, degenerative retinal damage, liver disease associated with chronic alcohol consumption, non-vascular gastrointestinal disorders, multiple sclerosis, muscular dystrophy and human cancers. In particular, compositions according to the present invention may be taken in addition to known or possible treatments for ALS (including FALS) Parkinson's disease and/or Alzheimer's disease.

Known treatments for Parkinson's disease include levodopa, selegiline and amantadine; dopamine agonists such as bromocriptine, lisuride, pergolide, cabergoline, talipexole, pramipexole and apomorphine; catechol-O-methyl-transferase (COMT) inhibitors such as tolcapone and entacapone; and anticholinergics such as trihexyphenidyl, procyclidine, benzatropine and orphenadrine.

Known treatments for Alzheimer's disease include cholinesterase inhibitors such as galantamine, rivastigmine, donepezil, and tacrine; and N-methyl D-aspartate (NMDA) antagonists such as memantine.

Currently, the only drug approved for use in the treatment of ALS is Rilutek® (2-amino-6-(trifluoromethoxy)benzothiazole; generic name Riluzole), which has been shown to produce a modest lengthening of survival in patients suffering from ALS. The mechanism of action of Riluzole is not known for certain. It is thought that Riluzole may reduce excitotoxicity by diminishing glutamate release. Side effects can include nausea, dizziness, weight loss, and elevatation in levels of liver enzymes.

Three drugs, Arimoclomol®, TRO19622 and Xaliproden hydrochloride, are also in development for the treatment of ALS.

Arimoclomol® is currently being developed by CytRx Corporation for use in the treatment of ALS and also Alzheimer's, Huntington's, and Parkinson's diseases. It is thought that Arimoclomol® stimulates the body's natural protein repair pathway by activating compounds called “molecular chaperones”, which assist proteins to achieve correct folding. As damaged and incorrectly folded proteins, called aggregates, are thought to play a role in many diseases, it is thought that activation of molecular chaperones could have a therapeutic efficacy for a range of diseases, including ALS.

TRO19622 (Cholest-4-en-3-one, oxime) is a cholesterol-like small molecule with neuroprotective properties, being developed by Trophos. TRO19622 has been shown to maintain survival of motor neurons in vitro, at levels comparable to neurotrophic factors.

Xaliproden hydrochloride (1-[2-(naphth-2-yl)ethyl]-4-(3-trifluoromethylphenyl)-1,2,5,6-tetrahydropyridine hydrochloride; SR 57746A) is being developed by Sanofi-Aventis. It is a serotonin 5-HT_{1A} receptor agonist, which appears to exhibit neurotrophic activities in vivo and in vitro.

The efficacy of the compositions and methods according to the present invention may be tested in studies along the lines set out below.

### ALS

### Model:

Rodents (rats and/or mice) bearing the mutation G93A on the SOD1 gene.

### Method:

8-10 animals are used per group. Animals in test groups are treated with one or more compositions according to the present invention (either force fed, or the compositions are included in their food) throughout the study.

Compositions comprise gliadin, and either SOD derived from wheat, which has an activity of 3000 IU per mg, or SOD derived from yeast, which has an activity of 1400 IU pet mg. In each case, the ratio of SOD to gliadin is approximately 36 IU of SOD per mg of gliadin, and in each case the final composition comprises approximately 35 IU/mg of composition, with gliadin comprising 98-99% of the weight of the composition. In addition, excipients may be included in compositions in order to improve stability. Table 1 provides further details of the gliadin and SOD for use in these compositions.

TABLE 1

| Source of SOD | | |
|---|---|---|
| | Wheat | Yeast |

| Activity/mg SOD | 3,000 | IU | 1,400 | IU |
|---|---|---|---|---|
| Weight of 36 IU SOD | 0.012 | mg | 0.026 | mg |
| Weight of gliadin/36 IU of SOD | 1 | mg | 1 | mg |
| Total weight of composition | 1.012 | mg | 1.026 | mg |
| (Gliadin + SOD)/36 IU | | | | |
| IU SOD/mg of composition | 35 | | 35 | |
| Total weight of composition | 28.1 | mg | 28.49 | |
| (Gliadin + SOD)/1000 IU | | | | |
| Weight of Gliadin (1000 IU) | 27.77 | mg | 27.77 | mg |
| Weight of SOD (1000 IU) | 0.33 | mg | 0.72 | mg |

The dose of composition administered to the rats is between 100 to 1000 IU/kg of body mass (i.e. 30 to 300 IU per rat and per day (when a rat is around 300 g)).

Treated animals bearing the mutation are compared to non-treated sibling animals bearing the mutation from the same litter.

Neurological symptoms (difficulties in moving, standing up, and turning over when placed on their back) in mutated animals generally appear at 2-3 months of age. Within a group of animals bearing the mutation, mortality typically commences at 100 days of age from birth, and all animals are dead by 120 days.

The principle assessment criterion of efficacy in the study is survival. Secondary objectives are to assess metabolic troubles, which tend to appear in mutated animals around 60 days from birth. Anomalies in energetic spending can lead to a loss of weight and decrease in fat reserve in the mutated animals, and this can be measured with a calorimetric chamber adapted for rodents. Finally, nuclear DNA resistance to exogenous oxidative stress (as a result of exposure to hydrogen peroxide (H₂O₂) is assessed (comet test on leucocytes nucleus DNA) for all groups.

### Expected Outcome:

It is expected that treated G93A mutated animals will, in comparison to G93A non-treated animals demonstrate:
An increase of 25% in life length;
A reduction in weight loss;
A reduction or normalisation of energetic spending; and/or
An increase of the resistance of the Leucocyte DNA to exogenous oxidative stress.

### Parkinson's Disease

### Model:

Various methods can be used to induce Patkinsonian-like features in animals. The inventors use treatment with rotenone in the present study.

### Method

Animals are divided into 3 groups, with 8-10 animals per group. One group of animals (“test group 1”) is treated with one or more compositions according to the present invention and then treated with rotenone; the second group of animals (“test group 2”) do not receive compositions according to the present invention, but are treated with rotenone; and the third group of animals (“control”) do not receive compositions according to the present invention and are not treated with rotenone.

Animals in test group 1 are treated with one or more compositions according to the present invention for 3 weeks. Compositions comprise gliadin, and either SOD derived from wheat, which has an activity of 3000 IU per mg, or SOD derived from yeast, which has an activity of 1400 IU per mg. In each case, the ratio of SOD to gliadin is approximately 36 IU of SOD per mg of gliadin, and in each case the final composition comprises approximately 35 IU/mg of composition, with gliadin comprising 98-99% of the weight of the composition. In addition, excipients may be included in compositions in order to improve stability. Table 1 provides further details of the gliadin and SOD for use in these compositions.

The dose of composition administered to the rats is between 100 to 1000 IU/kg of body mass (i.e. 30 to 300 IU per rat and per day (when a rat is around 300 g)).

Animals in test groups 1 and 2 are then injected with rotenone at levels known to induce Parkinsonian symptoms.

All animals are then assessed for 3-4 weeks following treatment using the following tests:
Water maze; and
Open field.

In addition, nuclear DNA resistance to exogenous oxidative stress (as a result of exposure to hydrogen peroxide (H₂O₂) is assessed (comet test on leucocytes nucleus DNA) for all groups.

### Expected Outcome:

It is expected that animals treated with compositions according to the present invention will, in comparison to non-treated comparable animals, have improved reactions in water maze and open field tests, and have more resistant DNA.

### Alzheimer's Disease

### Model:

Aged rodents, or rodents with accelerated aging as a result of exposure to ionizing radiation or a lack of selenium are used in the present study.

### Method:

8-10 animals are used per group. Animals in test groups are treated with one or more compositions according to the present invention for 8 weeks.

Compositions comprise gliadin, and either SOD derived from wheat, which has an activity of 3000 IU per mg, or SOD derived from yeast, which has an activity of 1400 IU per mg. In each case, the ratio of SOD to gliadin is approximately 36 IU of SOD per mg of gliadin, and in each case the final composition comprises approximately 35 IU/mg of composition, with gliadin comprising 98-99% of the weight of the composition. In addition, excipients may be included in compositions in order to improve stability. Table 1 provides further details of the gliadin and SOD for use in these compositions.

The dose of composition administered to the rats is between 100 to 1000 IU/kg of body mass (i.e. 30 to 300 IU per rat and per day (when a rat is around 300 g)).

In models using aged rats, test group animals are assessed in comparison to non-treated, but otherwise comparably aged animals.

In models wherein aging is induced (as a result of exposure to ionizing radiation or a lack of selenium) an additional control group is included in the study, comprising animals that have not been exposed to radiation, or restricted selenium, but are otherwise comparable.

The cognitive tests used are:
Water maze; and
Open field

Nuclear DNA resistance to exogenous oxidative stress (as a result of exposure to hydrogen peroxide (H₂O₂)) is assessed (comet test on leucocytes nucleus DNA) for all groups every 15 days.

### Phase II Study

Furthermore, an 18-month randomised double-blind placebo-controlled multi-centre, phase II study is to be conducted in humans. The primary objective of the study is to assess the efficacy of compositions according to the present invention as an add-on therapy to the use of riluzole in the treatment of probable or definite ALS, as compared to placebo. Efficacy is principally measured by 18-month survival rate. The secondary objective of the study is to assess the safety of compositions according to the present invention.

Approximately 400 patients are recruited via a number of centres in France. The recruitment period is approximately 4 months, with each centre recruiting 32-40 patients.

### Selection of Study Population

Patients are screened to assess suitability for inclusion in the study. The selection criteria for inclusion are as follows. The patient can be male or female; 18 to 80 years old; presenting with ALS defined as probable or definite according to the El Escorial criteria (revised); the ALS can be sporadic or familial; with bulbar or spinal onset; and symptoms of ALS must have been present for more than 6 months but less than 48 months; no gastrostomy, tracheostomy or non-invasive pulmonary ventilation (NIPV) current or pending; measurable Forced Vital Capacity (FVC), concordant after 3 measures at >50%; the patient must have been treated with riluzole at a stable dosage (50 mg b.i.d.) for at least 3 months; and must have given their written informed consent for inclusion in the study according to local law and regulations.

Exclusion criteria are as follows: known liver disease or renal insufficiency; aspartate aminotransferase (ASAT) and/or alanine aminotransferase (ALAT) serum levels ≧2 Upper Limits of Normal (ULN); currently evolving tumoral processes; evidence of major psychiatric disorder or clinically evident dementia precluding evaluation of symptoms; known hypersensitivity to any component of the study drugs or to other methylxanthines; the patient must not be pregnant or breast feeding; and if a female patient is of childbearing potential, the patient must use adequate contraceptive measures; a patient may not have participated in a clinical trial within the previous 3 months.

An inclusion visit to patients suitable for inclusion in the study takes place within 15 days of the screening visit. The inclusion day is defined as the randomization day.

### Study Design

Patients selected for inclusion in the study are randomized into two groups. One group receives an orally administered composition according to the present invention, as detailed below, the other group receives an orally administered placebo.

Patients receiving a composition according to the present invention will receive 1000 IU/day of SOD. Compositions comprise gliadin, in combination with SOD derived either from wheat origin (with a SOD activity of 3000 IU per mg of composition), or yeast origin (with a SOD activity of 1400 IU per mg of composition). In either case, the ratio of SOD to gliadin is approximately 35 IU of SOD per mg of gliadin, and in each case the final composition comprises approximately 35 IU/mg of composition, with gliadin comprising 98-99% of the weight of the composition. Compositions may, in addition, comprise gastro-resistant features (for example, micronisation of ingredients, inclusion of micronised gastro-resistant ingredients, or, more preferably, encapsulation in a gastro-resistant capsule). In addition, excipients may be included in compositions in order to improve stability. Table 1 provides further details of the gliadin and SOD which may be used in the compositions for administration.

Compositions according to the present invention are administered to the patient once a day, preferably before breakfast. The composition may be in the form of a controlled release tablet. If so, controlled release may be achieved by specific processing (for example micronisation) of the ingredients comprising the tablet, or, more preferably, encapsulation of the ingredients in a capsule, which may be a capsule comprising gastro-resistant ingredients. The placebo has the same administration schedule. Compositions according to the present invention, and placebos are identical in appearance and weight.

All patients involved in the study will be receiving the standard of care for ALS, including treatment with riluzole (which will not be supplied by the Study Sponsor). Patients must receive a 50 mg b.i.d. stable dosage of riluzole for at least 3 months to be included in the study. This dosage should be maintained over the double-blind study duration. Riluzole is typically taken morning and evening, within the 20 minutes prior to a meal. Change of riluzole dosage is allowed during the open-label study, but dosage, date of change and reason(s) for change are to be recorded in the Case Report Form (CRF).

Treatment with placebo/composition is continued for 18 months under double blind conditions.

### Patient Assessment During Study

Patients included in the study will receive follow-up visits every 3 months (±2 weeks) during the 18-month study duration (M3, M6, M9, M12, M15, M18), during which time the following information is recorded:
Status (death: yes/no).
Tracheostomy or non-invasive pulmonary ventilation (NIPV) (yes/no, date of event, reasons for tracheostomy or NIPV).
Concomitant treatments (d.c.i., dosage, dates of intake).
Physical examination (weight, blood pressure, heart rate).
Manual Muscle Testing (MMT)
Quality of Life Scale SF36 (Short form)
Laboratory examination: ALAT; ASAT; γGT (gamma-glutamyltransferase); total, conjugated and unconjugated bilirubinaemia; alkaline phosphatase (only if routinely performed in the centre); creatininaemia; alkaline reserve; complete blood count [CBC] and differential; platelet count, chloride (only if routinely performed in the centre), performed either at the centre attended by the patient, or, if the patient is of limited mobility, at the patient's home within 15 days prior to the following visit.
Compliance is assessed. Patients will be considered as compliant if the intake of assigned oral dosage forms is between 80% and 120%, as assessed by counting the returned blister packs.
Recording of Adverse Events (AEs).

Moreover, every month between scheduled visits, the patient's status (death: yes/no) is recorded by a study nurse as well as assessment of the patient's ALS Functional Rating Scale (FRS), by telephone. The telephone ALS-FRS assessment is always to be filled out by the same study nurse for the same patient.

Following the double-blind period of the study, open administration of compositions according to the present invention is to be allowed to patients until results of efficacy analysis are available.

### Assessment of Efficacy and Safety of Treatment

The primary efficacy criterion is the 18-month survival rate, together with respiratory status. Respiratory status is to be assessed, principally, on whether the patient is with or without invasive or non-invasive ventilation. The investigator will collect any death certificates and fill in a specific form in the CRF.

### Secondary Efficacy Criteria is Based Upon the Outcome of the Following:

The monthly ALS FRS questionnaires.
Manual Muscle Testing:
   upper limb strength
   lower limb strength
   neck
which will be graded as follows:
0: no contraction
1: flicker or trace contraction
2: active movement with gravity eliminated
3: active movement against gravity but not against resistance
4: active movement against gravity and resistance
5: normal power.
Quality of Life Scale SF 36: Patient will answer 36 questions covering the following domains:
   daily activity
   repercussions on physical health
   repercussions on psychological health
   physical activity
   bodily pain
   perceived health
   vitality (energy and tiredness)
   life and relationships with others (social activity)
   mental health.

The patient will be required to complete each item using a scoring system.
The occurrence of tracheostomy or NIPV is to be assessed using a specific questionnaire filled in by the investigator, in order to record the process leading to the decision to undertake artificial ventilation. Date of any event will be documented.

### Statistical Considerations

Sample Size Calculation

In order to be able to detect a 15% difference in survival rates at 18 months (from 40% on placebo to 55% on compositions according to the present invention, RR=0.65) between the 2 groups, with:
α=5%,
power=90%,
using a one-tailed Log-rank test
   362 patients (i.e. 181 patients in each group) are needed.

The inclusion of 400 patients in the study will allow detection of a difference of 10% with a 66% power and to detect an 8% difference with a 50% power.

Statistical Analysis

The details of the statistical analysis will be presented in a Statistical Analysis Plan, which will be issued before the set up of the Data Safety Monitoring Board.

An interim assessment of efficacy and safety data will be conducted at M12 by the Data Safety Monitoring Board, using Bayesian methods.

1. A pharmaceutical composition providing an oxidative signal upon administration to a subject, which triggers a therapeutic or prophylactic effect by priming the subject's body to combat the effects of oxidative stress.

2. A composition as claimed in claim 1, wherein the composition does not, upon administration, substantially increase levels of reactive oxygen species and/or does not substantially increase levels of oxidative stress.

3. A composition as claimed in claim 1, wherein the oxidative signal provided by the composition is hydrogen peroxide.

4. A composition according to claim 3, wherein the oxidative signal comprises an increase in the intracellular concentration of hydrogen peroxide.

5. A composition as claimed in claim 1, comprising an agent which is NADPH, NADH, superoxide dismutases, superoxide anions, succinate, choline, proline, malate, pyruvate, ketoglutarate, glycerol 3-phosphate, phorbol myristate acetate, antimycin A, antimycin, quinones, ubiquinone, rotenone, glycollate oxidase, D-amino acid oxidase, monoamine oxidases, oxidised natural anti-oxidants, or a combination thereof.

6. A composition as claimed in claim 5, wherein the agent is superoxide dismutase.

7. A composition as claimed in claim 5, wherein the agent has an activity of 50, 100, 200, 500, 800, 1000, 1200, 1500, 2000, 2200, 2500, 3000, 3500, 4000, 4500, 5000, 5500 or 6000 IU/mg

8. A composition as claimed in claim 5, comprising a minimal dose of 0.5, 1, 10, 50, 100, 200, 500, 1000, 1500, 2000, 2500, 2800, 2900, 3000, 3500, 4000, 4500, 5000, 5500, 6000, 6500, 7000, 8000, 9000, 10000, 10500 or 11000 IU of agent.

9. A composition as claimed in claim 1, further comprising a second component wherein said second component is selected from the group consisting of:
a) one or more naturally occurring oligosaccharides, prolamines, polymer films derived from prolamines, or a combination thereof;
b) one or more gastroresistant ingredients;
c) superoxide dismutase, gliadin and one or more gastroresistant ingredients; and
d) a pharmaceutically acceptable excipient, a neurotransmitter, one or more ions or any combination thereof.

10-12. (canceled)

13. A composition as claimed in claim 1, wherein administration is oral, nasal, by inhalation or injection.

14. A composition as claimed in claim 1, wherein the oxidative signalling provided by the composition is transmitted to immuno-competent cells within the subject.

15. A composition as claimed in claim 14, wherein the immuno-competent cells are in the gut wall.

16. A composition as claimed in claim 14, wherein the effect is transmitted by the immuno-competent cells to the entire organism.

17. (canceled)

18. A composition according to claim 1 for treatment of a disease, wherein the disease is Alzheimer's disease; Parkinson's disease; Lewy Body disease; cardiac disease; COPD; Down's syndrome; liver disease associated with chronic alcohol consumption; non-vascular gastrointestinal disorders; multiple sclerosis; muscular dystrophy, neuronal or cardiac injury resulting from ischemia/reperfusion.

19. A composition according to claim 18, wherein the disease is amyotrophic lateral sclerosis, and in particular amyotrophic lateral sclerosis associated with a mutation in Cu,Zn-SOD1.

20-31. (canceled)

32. A method of treating a disease in which oxidative stress is implicated in a subject, comprising administering to said subject a a composition that provides an oxidative signal to said subject, wherein said administering triggers a therapeutic or prophylactic effect in said subject by priming the body of said subject against oxidative stress.

33. The method of claim 32, wherein the composition does not, upon administration, substantially increase levels of reactive oxygen species and/or does not substantially increase levels of oxidative stress.

34. The method of claim 32, wherein the oxidative signal provided by the composition is hydrogen peroxide.

35. The method of claim 32, wherein the composition comprises an agent which is NADPH, NADH, superoxide dismutases, superoxide anions, succinate, choline, proline, malate, pyruvate, ketoglutarate, glycerol 3-phosphate, phorbol myristate acetate, antimycin A, antimycin, quinones, ubiquinone, rotenone, glycollate oxidase, D-amino acid oxidase, monoamine oxidases, oxidised natural anti-oxidants, or a combination thereof.

36. The method of claim 32, wherein said administering increases said subjects endogenous anti-oxidant defense.

37. The method of claim 32, wherein the effect triggered is an increase in the intracellular concentration of hydrogen peroxide.

38. The method of claim 37, wherein the amplification in intracellular concentration of hydrogen peroxide is in immuno-competent cells within the subject.

39. The method of claim 38, wherein the immuno-competent cells are in the gut of the subject.

40. The method of claim 32, wherein the administration of the composition to the subject upregulates the cellular hydrogen peroxide scavenging pathway.

41. The method of claim 32, wherein the effect of the treatment is transmitted by immuno-competent cells to the entire subject.

42. The method of claim 35, wherein the subject receives a minimal daily dose of 0.5, 1, 10, 50, 100, 200, 500, 1000, 1500, 2000, 2500, 2800, 2900, 3000, 3500, 4000, 4500, 5000, 5500, 6000, 6500, 7000, 8000, 9000, 10000, 10500 or 11000 IU of the agent.

43. The method of claim 32, further comprising treatment of the subject with medication or therapy prescribed for a pre-existing condition or disease.

44. The method of claim 43, wherein the pre-existing condition or disease is one in which oxidative stress is implicated.

45. The method of claim 43, wherein the condition or disease is probable or definite amyotrophic lateral sclerosis; Alzheimer's disease; Parkinson's disease; Lewy Body disease; cardiac disease; COPD; Down's syndrome; liver disease associated with chronic alcohol consumption; non-vascular gastrointestinal disorders; multiple sclerosis; muscular dystrophy, neuronal or cardiac injury resulting from ischemia/reperfusion.

46. The method of claim 43, wherein the disease is amyotrophic lateral sclerosis, and in particular amyotrophic lateral sclerosis associated with a mutation in Cu,Zn-SOD1.
